(19) United States
(12) Patent Application Publication
(10) Pub. No.: US 2025/0353009 A1
(43) Pub. Date: Nov. 20, 2025

(54) SYSTEM, METHOD AND DEVICE FOR A MICROFLUIDIC ASSAY

(71) Applicant: Prodia Technologies, Inc., Haines City, FL (US)

(72) Inventor: Ismael Marvin Voltaire, Hollywood, FL (US)

(21) Appl. No.: 19/209,717

(22) Filed: May 15, 2025

Related U.S. Application Data
(60) Provisional application No. 63/648,236, filed on May 16, 2024.

(51) Int. Cl. B01L 3/00; A61B 5/00; A61B 10/02; G16H 40/67

(52) U.S. Cl. CPC B01L 3/502753; A61B 5/6802; A61B 10/0233; G16H 40/67; B01L 2200/0621; B01L 2200/16; B01L 2300/0819; B01L 2300/12; B01L 2400/049

(57) ABSTRACT

An exemplary microfluidic assay device is disclosed that enables minimally invasive extraction and detection of biomarkers in biological fluids. The device includes microneedles for fluid collection, a conductive substrate functionalized with aptamers for biomarker binding, and internal structures to regulate fluid flow. The system supports repeated use through cleaning mechanisms that remove bound biomarkers from the aptamers using heat or chemical reagents. In some embodiments, valves are pressure-activated by a vacuum and control the timing of reagent release from internal reservoirs. Aptamers may be detached and replaced through sequential reagent flows, allowing refunctionalization of the sensor surface for continued use.

### CROSS-REFERENCE TO RELATED APPLICATIONS

This application claims priority to U.S. Provisional Patent Application No. 63/648,236, filed May 16, 2024, the entire contents of which are hereby incorporated by reference.

### FIELD OF THE INVENTION

The present disclosure relates generally to medical diagnostics and, more particularly, to a system, method, and device for a reusable, self-cleaning microfluidic assay (100) configured to detect biomarkers in interstitial fluid or blood using aptamer-based biosensors.

### BACKGROUND

Individuals with chronic conditions can benefit from frequent and thorough blood tests or biomarker measurements. For example, frequent blood draws can closely track and manage various health conditions, thus enabling timely clinical intervention from healthcare experts. As a result, better health outcomes can be achieved, and there can be a decrease in early mortality.

Products used for biomarker measurements are often invasive and require healthcare staff to take samples and analyze them in a clinical laboratory. This process can be time-consuming, costly, and inconvenient for patients. Furthermore, traditional blood tests may not provide real-time monitoring and are not designed for continuous use. Disposable tests are more portable and provide rapid results, but they may not be as accurate as standard laboratory tests.

There remains a need for a minimally invasive, accurate, and reusable diagnostic device capable of autonomous operation and real-time biomarker monitoring outside clinical settings.

### SUMMARY OF THE INVENTION

The proposed technology is a biochip (100) that aims to overcome these disadvantages by providing a minimally invasive, reusable, and self-cleaning solution that allows patients to collect samples and measure biomarkers on their own, providing accurate and real-time results.

The present disclosure relates generally to a microfluidic assay (100), and more specifically, to exemplary embodiments of exemplary system, method, computer-accessible medium, and circuit for a microfluidic assay (100) for the extraction and analysis of interstitial fluid or blood. The exemplary microfluidic assay (100) can include microneedles for extracting the fluid, a plate of conductive material, and aptamers that can bind with biomarkers to detect the presence of the same. The microfluidic assay (100) can be cleaned by removing biomarkers from aptamers that were bound together in previous tests. In an exemplary microfluidic assay (100), bound biomarkers can be so removed through heat or reagents. In one example, the valves of the biochip (100) can be pressure-activated by a vacuum. In one example, the valves can regulate the timing of fluid release from the reservoirs. The aptamers can be removed from a gold plate through appropriate reagents, and new aptamers can be reattached via a sequential release of reagents that link them to the gold plate, carbon plate, or a conductive polymer.

In one example, the microfluidic assay (100) can be used as a Gene Identification biosensor, which employs complementary strands of a mutant gene sequence. The binding of the sequence can result in a weight and frequency change. The exemplary microfluidic assay (100) can offer significant advantages over existing technologies and can be readily adapted for various medical and diagnostic applications.

An exemplary system, method, computer-accessible medium, and circuit for a microfluidic assay (100) can collect frequency data from the microfluidic assay (100) and convert it into live data. In one example, the intervals of data collection can be as short as one second (or shorter) when the device is turned on and is actively tracking the data. The user can obtain the data as it becomes available or on demand. In one example, the data can represent a concentration of the protein in blood or interstitial fluid. In one example, the data can be transmitted through an ESP8266 IoT chip to Firebase.

In one example, the transmitted data can be used for further analysis. For example, concentration readings can be compared to the patient's baseline, where the baseline is established by the physician right before the patient leaves the hospital in the remote monitoring application of the exemplary embodiment of the present disclosure. As another example, a user's baseline can be established after several uses of the device according to the present disclosure, and thus, creating a pattern of normal ranges for the user. As yet another example, population averages that take into account the users' age, gender, weight, and other health factors can be analyzed to establish a user's baselines. In one example, the user's blood test data can be aggregated with the health sensor data to create a holistic score of the user's total health. In one example, when a concentration change is more than a threshold, there can be an indication of an acute health episode. The exemplary system, method, computer-accessible medium, and circuit for the microfluidic assay (100) can notify a physician immediately, where the physician can intervene.

An exemplary system, method, computer-accessible medium, and circuit for a microfluidic assay (100) can enable users to easily insert new chips that already harbored new biosensors. These chips can repurpose a watch's function to read and send test results. Additionally, the microfluidic assay (100) can integrate disposable microneedles and reagents, further simplifying the process for obtaining blood or interstitial fluid from users. The exemplary microfluidic assay (100) accordingly the embodiments of the present disclosure can, among others, address the challenges of microneedles, reagents, and biosensors in a wearable device (300).

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIGS. 1-8 show exemplary views of an exemplary microfluidic assay (100) according to an exemplary embodiment of the present disclosure.

FIGS. 9-11 show exemplary views of an exemplary actuator (170) according to an exemplary embodiment of the present disclosure.

FIGS. 12-13 show exemplary views of an exemplary microfluidic assay (100) and a wearable device (300) according to an exemplary embodiment of the present disclosure.

FIG. 14 shows an exemplary physician facing interface according to an exemplary embodiment of the present disclosure.

FIG. 15 is an exemplary flow chart for using the exemplary microfluidic assay (100) of the present disclosure.

FIGS. 16A and 16B show top and side views, respectively, of an exemplary biochip casing. FIG. 16A illustrates the QCM sensor cavity (260), access channels, and micropore array. FIG. 16B shows the positioning of the microneedle array (120) and a compression ridge.

FIGS. 17A and 17B show bottom perspective views of the microneedle array (120). FIG. 17A presents the full microneedle array in isometric view, and FIG. 17B offers a zoomed-in view of several individual microneedles.

FIGS. 18A and 18B illustrate details of a single microneedle. FIG. 18A shows the overall shape with a sharpened tip and micropore (270), and FIG. 18B depicts the base diameter which may be approximately 0.38 mm.

FIG. 19 shows a side view of an individual microneedle (120) which may have a height of approximately 2.0 mm.

FIG. 20 is a sectional view of the biochip structure showing the connection between micropores (270) and internal microchannels (280) in a microneedle (120).

FIG. 21 shows a top view of the micropore array within the biosensor region or cavity (260).

FIG. 22 shows a zoomed-in view of a single micropore structure (270) with a microchannel (280) opening.

### DETAILED DESCRIPTION

Exemplary embodiments of the invention will now be described in order to illustrate various features of the invention. The embodiments described herein are not intended to be limiting as to the scope of the invention, but rather are intended to provide examples of the components, use, and operation of the invention.

In this disclosure, the terms microfluidic assay (100), lab on a chip, biochip (100), or device has been used interchangeably.

An exemplary system, method, computer-accessible medium, and circuit for a microfluidic assay (100) can include an inlet (150) and an outlet (140) on either side of the biochip (100) that connects to the tubing in a wearable device (300). The tubing can be controlled by the vacuum. The vacuum can be, e.g., a peristaltic vacuum that can then have a connection to the microfluidic assay (100). When the microfluidic assay (100) is connected and the vacuum is on, the blood or interstitial fluid can travel through the microneedles and onto the microfluidic assay (100) for piezoelectric detection. A combination of air filled reservoirs along with reagent filled reservoirs can allow for time sensitive release of reagents at a consistent pressure. In one example, pressurized valves can also allow for greater control over fluid release. The fluid can then travel through the device and return to the biochip (100) via the inlet (150) connection between the device and the biochip (100). The fluid would then go to the waste compartment of the device. The reagents can follow the same flow of movement. Therefore, in one example, the movement of microfluidics can be facilitated by the connection of the microfluidic assay (100) to, e.g., a wearable device (300) where the vacuum will facilitate movement.

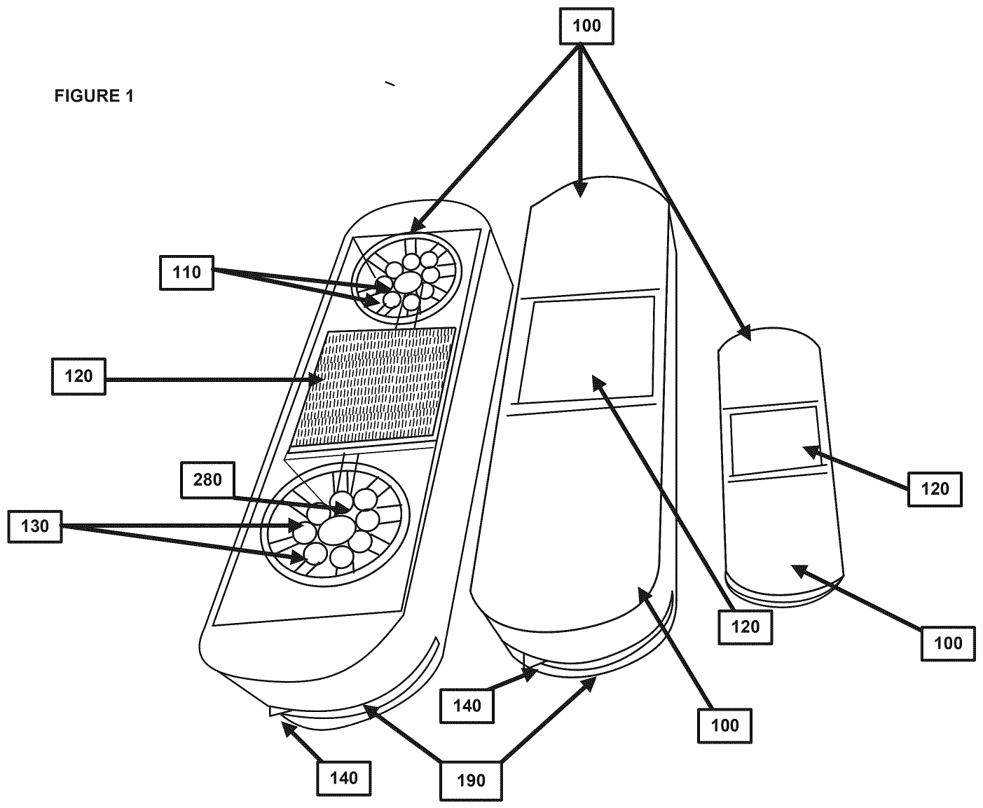

FIG. 1 illustrates a top view of an exemplary microfluidic assay (100) device (100), also referred to as a biochip (100), in both the open and closed positions according to one embodiment of the present disclosure. The biochip (100) includes at least one, and may include a plurality, of: reagent reservoirs (110), microneedles (120), and a biosensor cavity or cavities (also called biosensor region or regions) (130). These components may be integrated into a multilayer microfluidic structure designed for sample acquisition, analyte detection, and internal fluid routing.

The reagent reservoirs (110) may be arranged in alternating fashion along the body of the biochip (100). In one example, there are eight total reservoirs, with every other reservoir configured to hold air and the remainder configured to hold liquid reagents. In certain embodiments, each reagent reservoir (110) may capable of holding at least approximately 45 microliters of fluid. The reservoirs may be formed in stacked layers using microfabrication techniques suitable for polymeric substrates such as PMMA (polymethyl methacrylate).

In certain embodiments, the biosensor region(s) (130) may be configured to receive fluid samples extracted via the microneedles (120) and hold(s) a test volume of approximately 45 microliters. The biosensor may be functionalized with aptamers or other selective binding agents, enabling detection of one or more biomarkers in the extracted fluid. In the illustrated embodiment, the biosensor is configured to test for eight biomarkers simultaneously.

In an exemplary embodiment, refunctionalization reagents stored in designated reagent reservoirs (110) may be released into the biosensor region (130) in a timed sequence controlled by the pressure-actuated valves. For example, a cleaning reagent such as piranha solution may be introduced first to remove residual aptamers from the conductive surface. A subsequent flush with buffer or neutralizing fluid may follow. Then, a solution containing a binding agent such as avidin may be delivered to facilitate attachment of new aptamers introduced in a final reagent pulse. The sequence, timing, and volumes of these reagents may be preprogrammed or controlled by the wearable device's onboard processor. These reagents are provided as illustrative examples and are not intended to limit the scope of the present disclosure.

Microneedles (120) are located at the bottom surface of the biochip (100) and may be formed from biocompatible materials such as PMMA or polyimide. These microneedles are configured to penetrate the stratum corneum and access interstitial fluid or capillary blood in a minimally invasive manner. Outlet valves (140) at the end of outlet tubing (190) on either side of the biochip (100) may be configured to connect to the tubing in a wearable device (300).

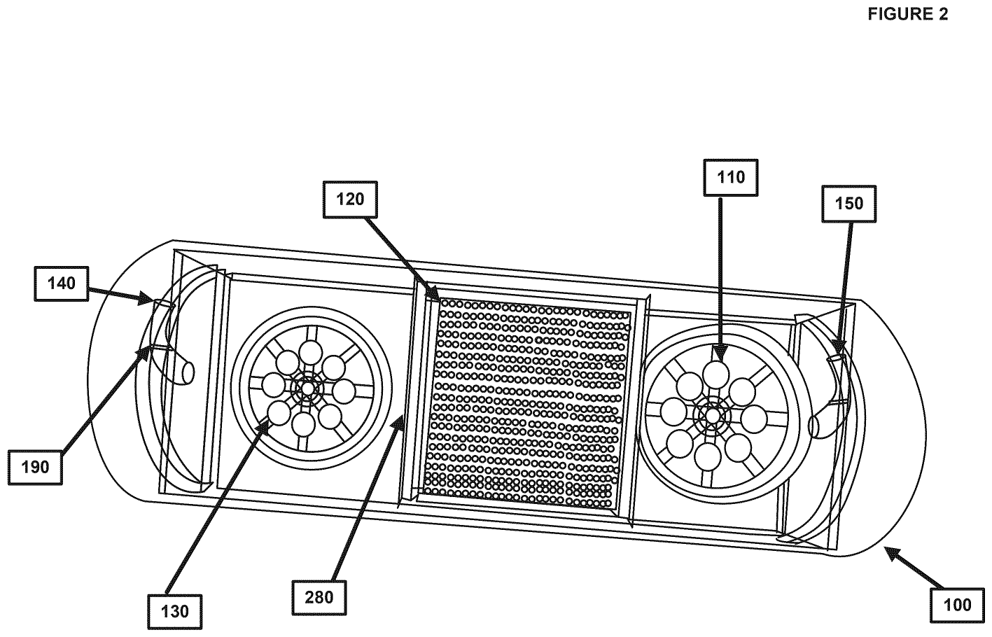

FIG. 2 illustrates a schematic fluid flow diagram through the microfluidic assay (100) device (100), according to one embodiment of the present disclosure. The figure depicts the directionality and arrangement of fluid pathways connecting the microneedles (120), biosensor region (130), outlet (140), and inlet (150).

As shown in FIG. 2, biological fluid may enter the biochip (100) through the microneedles (120), which may be configured to access interstitial fluid or blood from the skin surface. The extracted fluid may be directed through internal microchannels toward a biosensor region (130), where biomarker analysis may be performed.

After analysis, fluid may exit the biosensor region (130) via the outlet (140) at the end of the outlet tube (190), which may be located on one lateral side of the biochip (100), as is depicted. From there, fluid may be directed externally for disposal or recirculation, depending on the operational mode of the device.

In some embodiments, fluid may reenter the biochip (100) through an inlet (150) located opposite the outlet (140), enabling closed-loop circulation or cleaning cycles. The inlet (150) is in fluid communication with one or more internal chambers or reservoirs that may store reagents, buffer solutions, or clean samples for reanalysis or system flushing.

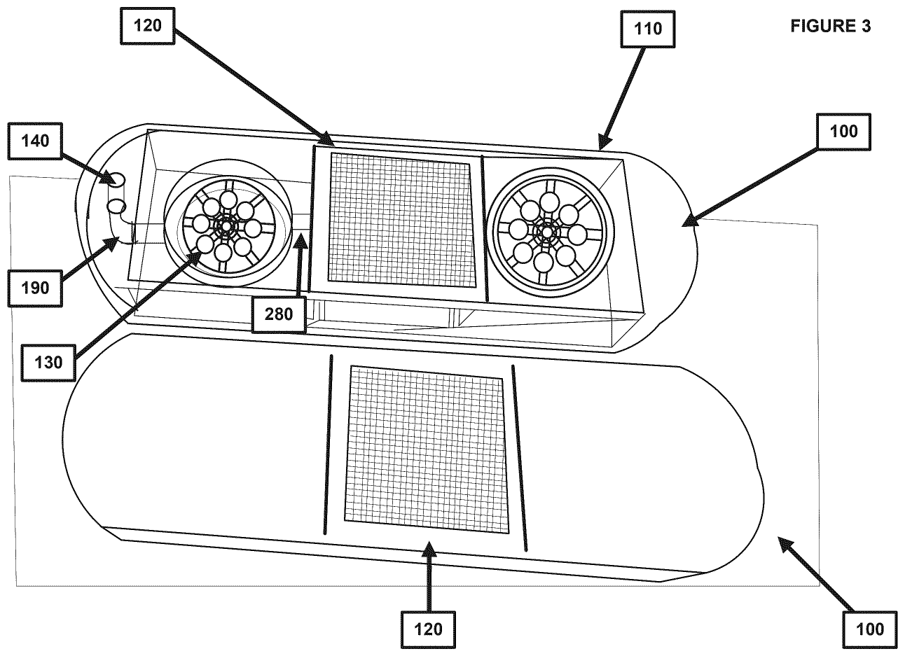

FIG. 3 illustrates another view of the microfluidic assay device (100) which may interface with an external microcontroller (160) (not depicted) in the wearable device (300) which external microcontroller (160) may also be referred to as the PCB or IoT chip component or wireless communication module (160), according to one embodiment of the present disclosure. The microcontroller chip (160) may be integrated within the wearable device (300) and may be configured to interface with the biosensor electrode embedded in the biochip (100). Such a connection enables the acquisition and interpretation of data generated by the biosensor (130).

The connection between the microcontroller (160) and the biosensor electrode may occur through a set of electrical contacts or pins located at the bottom of the biochip (100), which align with corresponding contacts in the wearable device (300). When the biochip (100) is inserted into the wearable device (300), these contacts allow electrical signals from the biosensor (130) to be transmitted to the microcontroller (160) for processing.

The microcontroller (160) may be configured to receive and interpret electrical signals generated by the binding events between aptamers and target biomarkers in the biosensor region (130). These signals can then be converted into digital data representing the concentration or presence of specific biomarkers in the sample fluid. The microcontroller (160) may further process the data and transmit it to a user interface or external application for display, storage, or clinical analysis.

In some embodiments, the connection points between the biochip (100) and the microcontroller (160) may also support bidirectional communication, enabling commands or calibration signals to be sent from the wearable device (300) to the biochip (100) during operation.

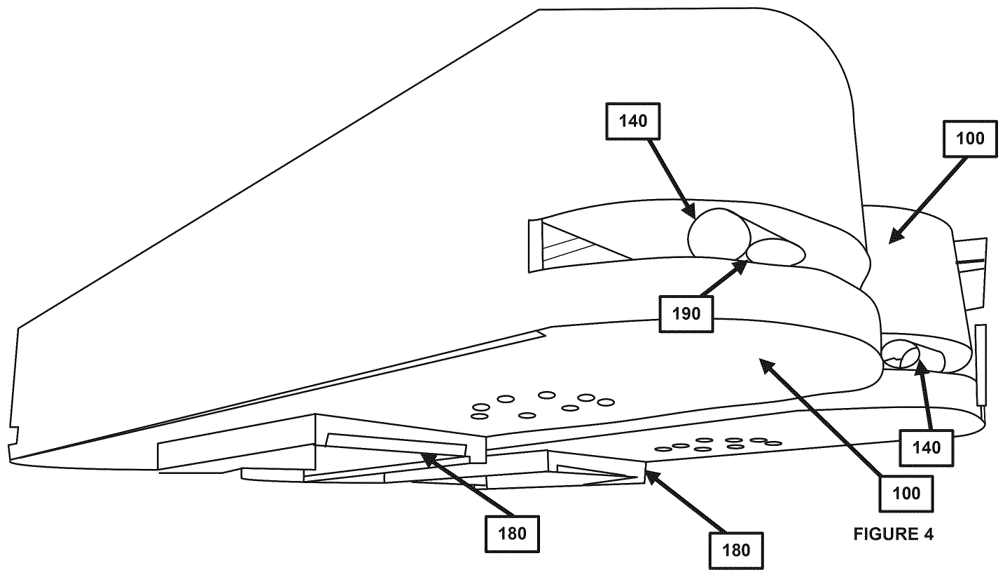

FIG. 4 illustrates an exploded view of the portion of the microfluidic assay device (100) that can act as the mechanical and fluidic interface between the microfluidic assay device (100) and the wearable device (300) according to one embodiment of the present disclosure. This figure highlights the structural integration that allows the biochip (100) to be locked into place within the device and actuated for fluid collection.

The biochip (100) includes an extension (180) located beneath the microneedle (120) array (120). This extension (180) may be configured to engage with a locking feature in the wearable device (300), securing the biochip (100) in the proper position for use. The extension (180) also defines an opening through which the actuator (170) (not depicted in FIG. 4) can deliver a controlled mechanical force to drive the microneedles (120) into the skin.

Once the biochip (100) is secured within the device, the actuator (170) is configured to strike upward through the opening in the extension (180), pushing the microneedles (120) into the skin surface in a controlled and minimally invasive manner. This actuation enables the collection of interstitial fluid or blood, which is then routed through the microfluidic channels toward the biosensor region (130).

The figure further illustrates the outlet (140) of the outlet tube (190), which is fluidly connected to a vacuum source within the wearable device (300). The vacuum draws fluid from the microneedles (120), into a chamber beneath the microneedles, and through the biosensor (130). After analysis, the fluid may exit through the outlet (140) located below the biochip (100). This vacuum-assisted flow pathway allows for precise control over fluid movement within the microfluidic assay device (100).

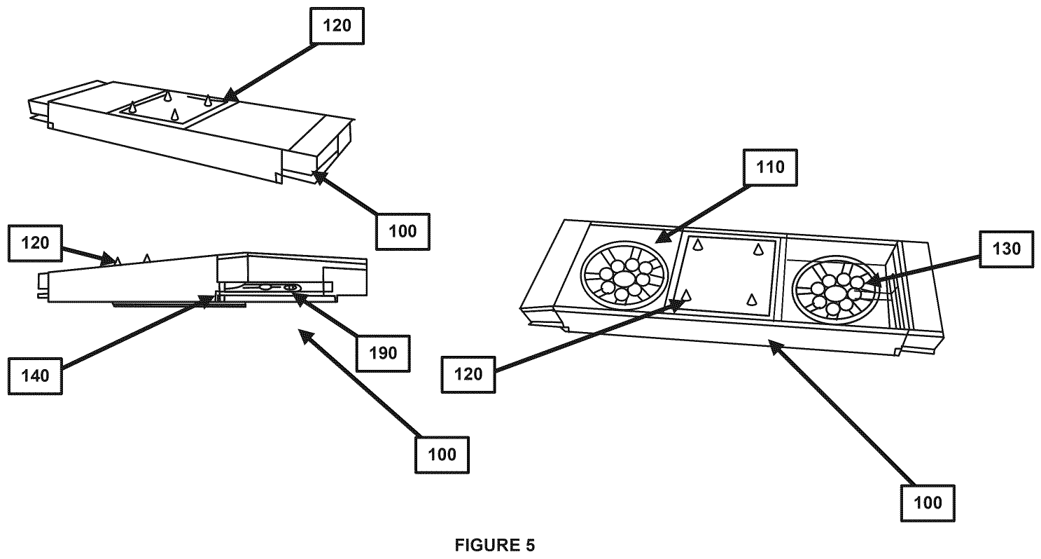

FIG. 5 illustrates an exemplary biochip (100) according to one embodiment of the present disclosure. The figure shows a layered arrangement including microneedles (120) for fluid collection, reagent reservoirs (110), and a biosensor region (130) configured to test for multiple biomarkers.

In the illustrated embodiment, the microneedles (120) collect small volumes of interstitial fluid or blood from the user. The extracted fluid is directed into the biosensor region (130), where detection is carried out using aptamer-functionalized surfaces.

The figure also depicts internal reagent reservoirs (110) that store fluids used to sanitize and refunctionalize the sensor for repeated use. These reagents may be selectively released to clean the sensor and prepare it for a new test cycle.

The biochip (100) is designed to be removable and disposable after a fixed number of uses. In one example, the biosensor region (130) is configured to test up to five biomarkers simultaneously and to complete multiple test cycles before disposal.

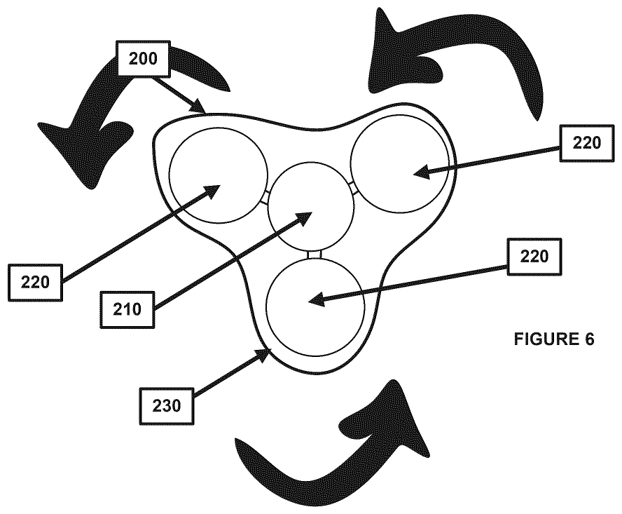

FIG. 6 illustrates an alternative vacuum mechanism (200) for driving fluid flow within the microfluidic assay (100) system, according to one embodiment of the present disclosure. This mechanism may be based on a microscale peristaltic pump configuration that uses electrostatic repulsion to generate rotational motion and fluid displacement.

The mechanism may include a centrally positioned negatively charged metal cylinder (210), to which an electric potential may be applied. This cylinder (210) may be surrounded by three additional negatively charged cylinders (220), each mechanically coupled to the central cylinder via an axis such that when electricity is applied to the central cylinder, it repels the three outer cylinders (220), and their orientation and mutual repulsion induce rotational motion.

The rotating cylinders (220) may be enclosed by flexible tubing (230) that contains fluid. As the cylinders turn, they compress the tubing in a sequential manner, thereby generating peristaltic movement of the fluid through the tubing. This allows fluid to be pumped without requiring direct contact with internal regions of the microfluidic assay (100) device itself.

In some embodiments, this rotational vacuum mechanism (200) may be implemented at the millimeter scale (or smaller) as part of a self-contained fluid control subsystem within the wearable device (300). This design provides an alternative to traditional diaphragm- or piston-based vacuum mechanisms and may simplify device architecture or reduce energy consumption in certain applications. The alternative vacuum mechanism (200) may be located in physical contact with the outlet tube (190) near the outlet (140) of the outlet tube (190).

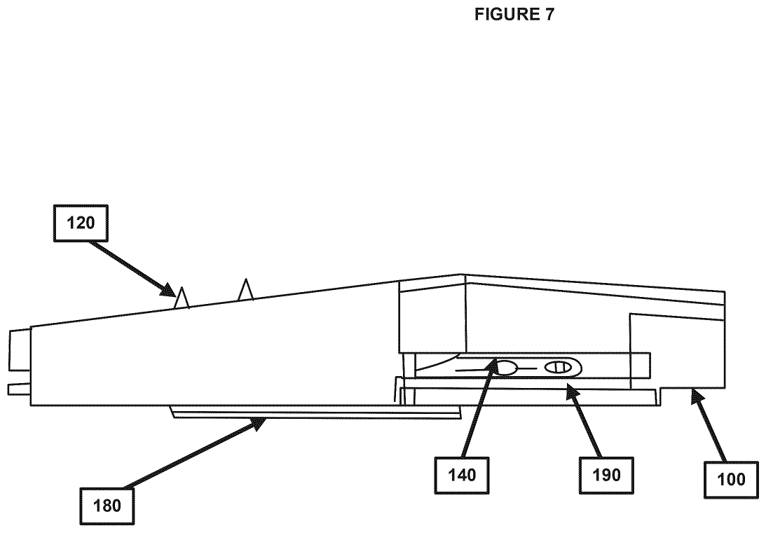

FIG. 7 illustrates is a representation of the microfluidic assay device (100), according to one embodiment of the present disclosure.

The biochip (100) includes lateral tubing connections on each side—an outlet (140) on one side and an inlet (150) on the opposite side (not shown). The outlet (140) serves as the exit point for fluid that has passed through the biosensor region (130), while the inlet (150) serves to reintroduce fluid into the device.

In one embodiment, the inlet (150) is positioned adjacent to the biosensor region (130), which contains aptamers or other molecular recognition elements. When the biosensor completes analysis and determines the concentration of a target biomarker, the vacuum system is activated.

The vacuum draws fluid out of the biochip (100) through the outlet (140), and subsequently returns it to the biochip (100) through the inlet (150). This closed-loop configuration allows for controlled removal, recirculation, or flushing of fluid through the assay system, and may support additional cleaning or calibration cycles following biomarker detection.

FIG. 8 illustrates an alternative fluid circulation mechanism for the microfluidic assay device (100), wherein a rotating peristaltic vacuum may be used to apply pressure to a malleable diaphragm, according to one embodiment of the present disclosure.

In this embodiment, the vacuum mechanism (200) rotates across a diaphragm composed of polydimethylsiloxane (PDMS), a flexible and biocompatible polymer. The diaphragm is positioned in fluid communication with the internal microchannels of the biochip (100).

As the rotating vacuum applies localized pressure across the PDMS diaphragm, it induces displacement of fluid within the internal assay structure. This configuration allows for controlled movement of fluid without requiring a direct physical connection between the outlet (140) and inlet (150) tubing ends of the biochip (100).

This design provides an alternative to full closed-loop tubing configurations. Instead of circulating fluid through external tubing and returning it to the inlet (150) on the waste side of the device, the PDMS diaphragm-based mechanism moves fluid entirely within the internal architecture of the biochip (100) through vacuum-assisted actuation.

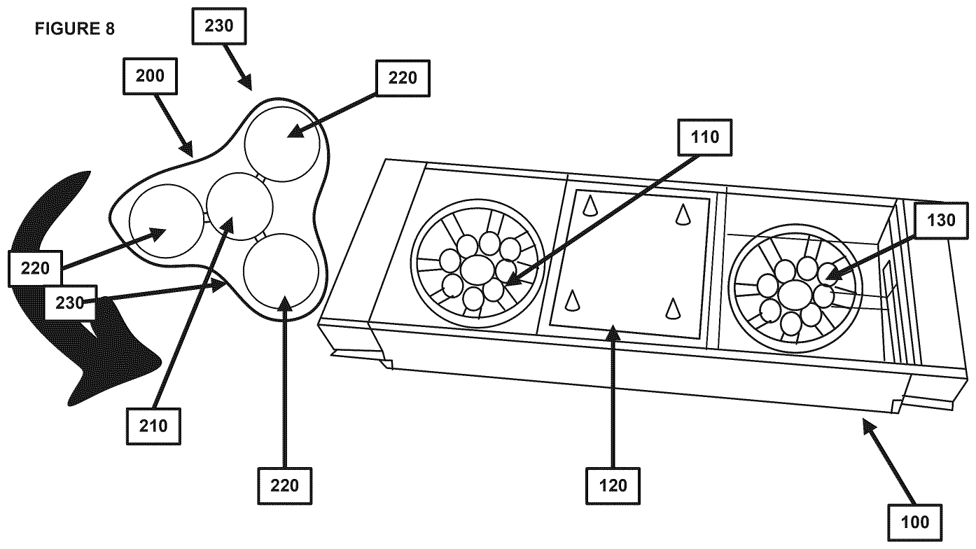

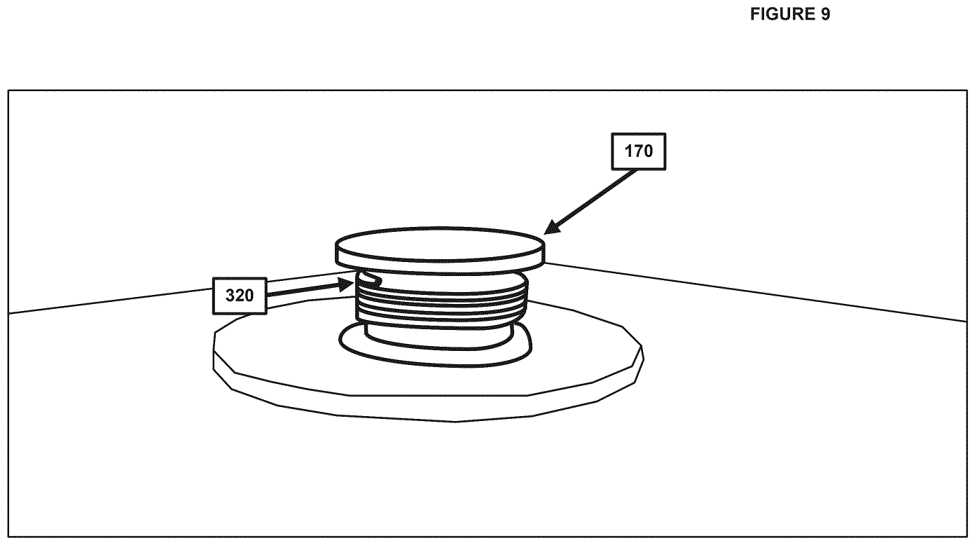

FIG. 9 illustrates one embodiment of a mechanical actuator (170) configured to deploy the microneedles (120) of the microfluidic assay device (100), according to one embodiment of the present disclosure. The figure depicts an actuator (170) in its undeployed, pre-engagement state.

In the illustrated embodiment, pressing the bottom of the actuator (170) causes an internal plunger or striking element to move upward. This component may be aligned to strike directly (or indirectly) beneath the microneedle (120) portion of the biochip (100), pushing the microneedles into the skin in a controlled, minimally invasive manner. The actuator (170) may contain an internal spring mechanism (320). The spring may be configured to release stored energy and deliver an upward strike against the underside of the biochip (100), thereby deploying the microneedles (120) into the skin.

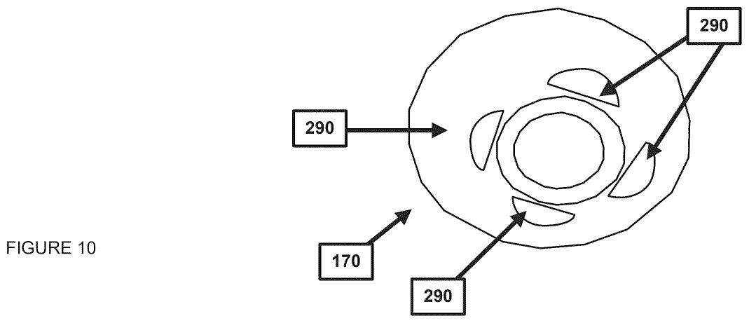

FIG. 10 illustrates a perspective view of the mechanical actuator (170) housed within the wearable device (300), according to one embodiment of the present disclosure. This figure highlights the physical interaction points and internal mechanism used to activate the microneedle (120) deployment.

The bottom of the device may include grooves or indentations that serve as grip points for the user (290). These grooves allow the user to manually rotate the actuator (170) housing. Rotating the actuator (170) may be used as a mechanism to cause the actuator to lower into position beneath the microneedle (120) portion of the biochip (100).

Once lowered (in embodiments where lowering is necessary) the user can press the actuator (170), which may contain an internal spring mechanism (320). The spring may be configured to release stored energy and deliver an upward strike against the underside of the biochip (100), thereby deploying the microneedles (120) into the skin.

This manual rotation and spring-loaded striking system provides a simple and controlled mechanism for microneedle (120) insertion, enabling safe and repeatable actuation without the need for powered components.

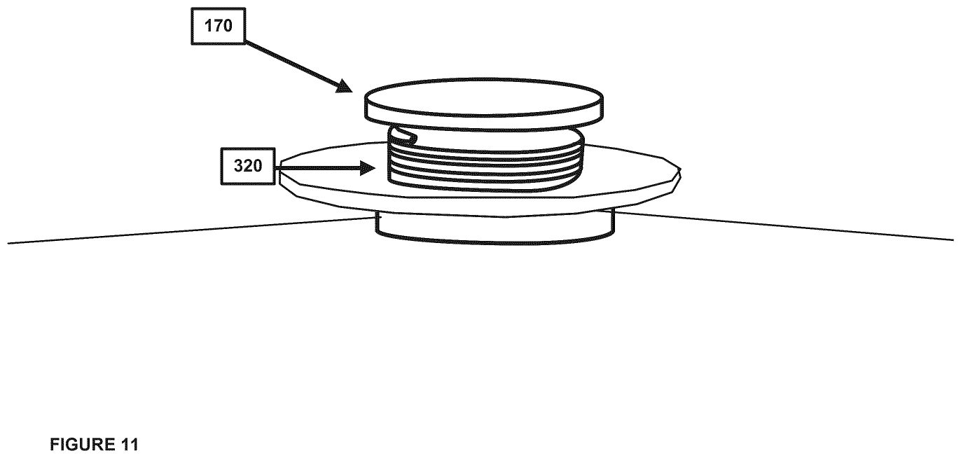

FIG. 11 illustrates a functional diagram of the actuator (170) mechanism (170) in the engaged or activated position, according to one embodiment of the present disclosure. The figure shows how manual interaction with the base of the wearable device (300) triggers microneedle (120) deployment.

When the user turns the indented grooves at the bottom of the device, the actuator (170) is lowered into position beneath the microneedles (120) of the biochip (100). Once aligned, the user presses the actuator (170), initiating a mechanical strike.

Upon being pressed, the actuator (170), shown in orange in the figure, exerts an upward force on the underside of the microneedles (120), causing them to extend through the device and penetrate the user's skin. This controlled actuation facilitates minimally invasive fluid collection from the skin surface.

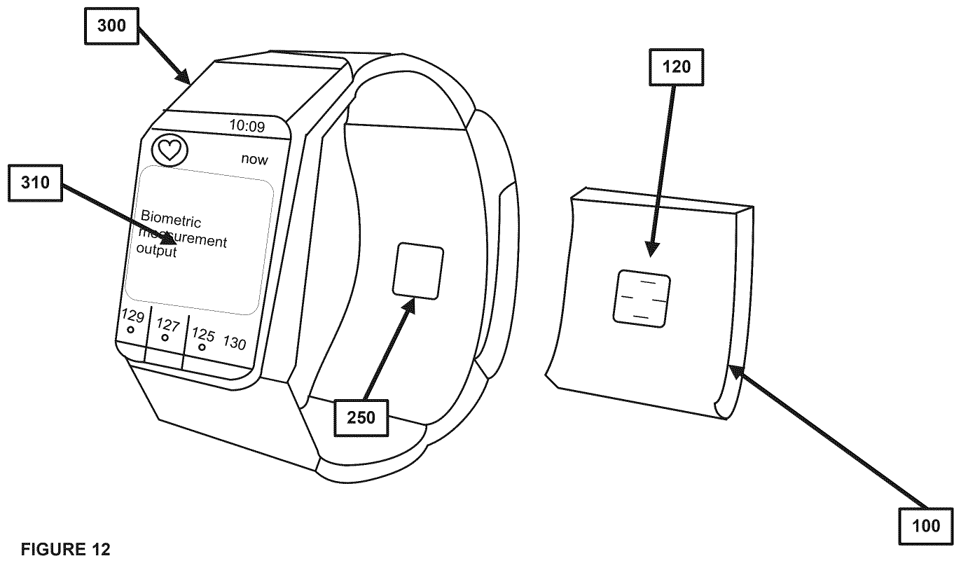

FIG. 12 illustrates a perspective view of a wearable diagnostic system incorporating the microfluidic assay (100) device (100), according to one embodiment of the present disclosure. The figure shows how the disposable biochip (100) may physically integrate with a reusable wearable housing for fluid collection and biomarker detection.

The wearable device (300) includes a slot (240) configured to receive and securely hold the biochip (100). The slot (240) is shaped to ensure correct orientation and alignment, allowing for reliable contact with the device's internal actuator (170), vacuum system, and electrical interface components.

The housing may further includes a microneedle (120) interface hole (250) aligned beneath the slot (240). When the biochip (100) is inserted, the microneedle (120) array (120) is positioned directly over the microneedle (120) interface hole (250), allowing the microneedles to extend through the opening and penetrate the user's skin during use. This arrangement is one means to enable minimally invasive access to interstitial fluid or blood while maintaining the protective enclosure of the remaining components of the biochip (100).

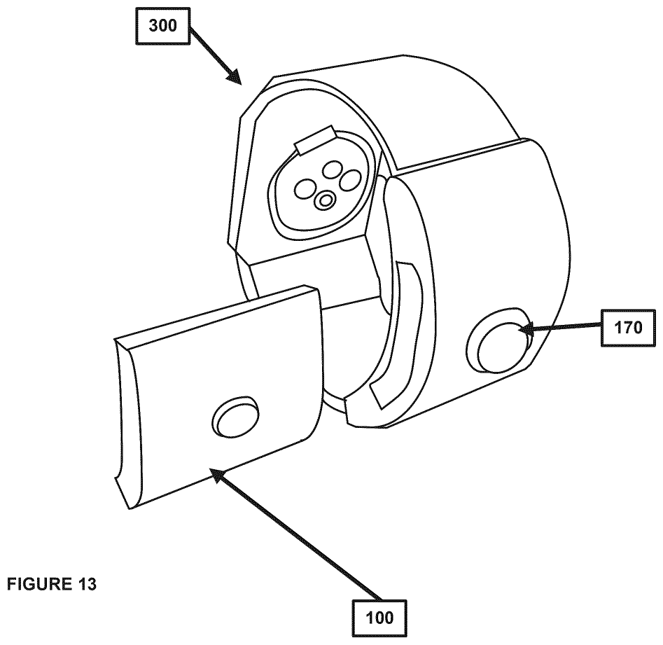

FIG. 13 illustrates a side view of the wearable diagnostic system with the microfluidic assay (100) device (100) inserted into the housing, according to one embodiment of the present disclosure. This view highlights the orientation and placement of the biochip (100) within the device, as well as the actuator's (170) position relative to the microneedle (120) array (120).

The wearable housing may include a slot (240) into which the biochip (100) may be inserted. In the illustrated configuration, the biochip (100) is shown seated within the slot (240), with its microneedle (120) array (120) positioned above the actuator (170). The actuator (170) is located within the lower portion of the housing and is aligned to deliver a vertical force when triggered.

When the actuator (170) is pressed, it may move upward to engage the underside of the microneedle (120) region of the biochip (100), pushing the microneedles (120) toward the user's skin or tissue. While the opening through which the microneedles pass is not shown in this view, the figure emphasizes internal alignment and component positioning useful for proper actuation and sampling.

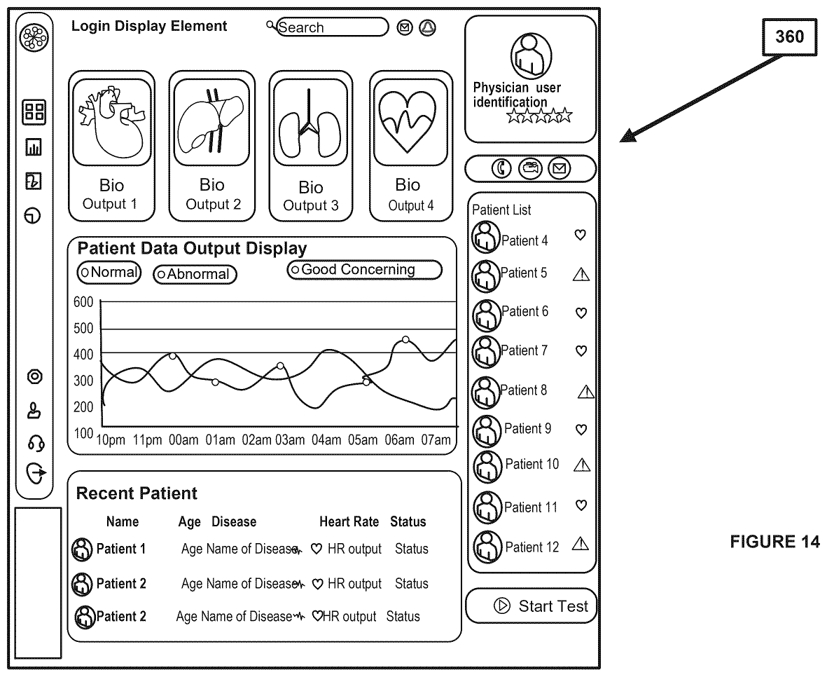

FIG. 14 illustrates an exemplary physician-facing interface for reviewing patient health data collected by the wearable diagnostic system, according to one embodiment of the present disclosure. This dashboard provides healthcare professionals with real-time access to biomarker readings and trends derived from the microfluidic assay device (100).

The interface may display concentration data for specific biomarkers, recent sampling times, and longitudinal graphs tracking changes over time. In some embodiments, the dashboard may also include personalized thresholds, historical comparisons, and visual alerts when biomarker levels exceed predefined limits.

The physician dashboard (360) may be accessed through a secure online portal or integrated electronic health record (EHR) system. Data presented on the dashboard is transmitted from the wearable device (300) via a wireless communication module (160), such as an IoT chip, and stored on a cloud-based platform or hospital server for clinician review and intervention.

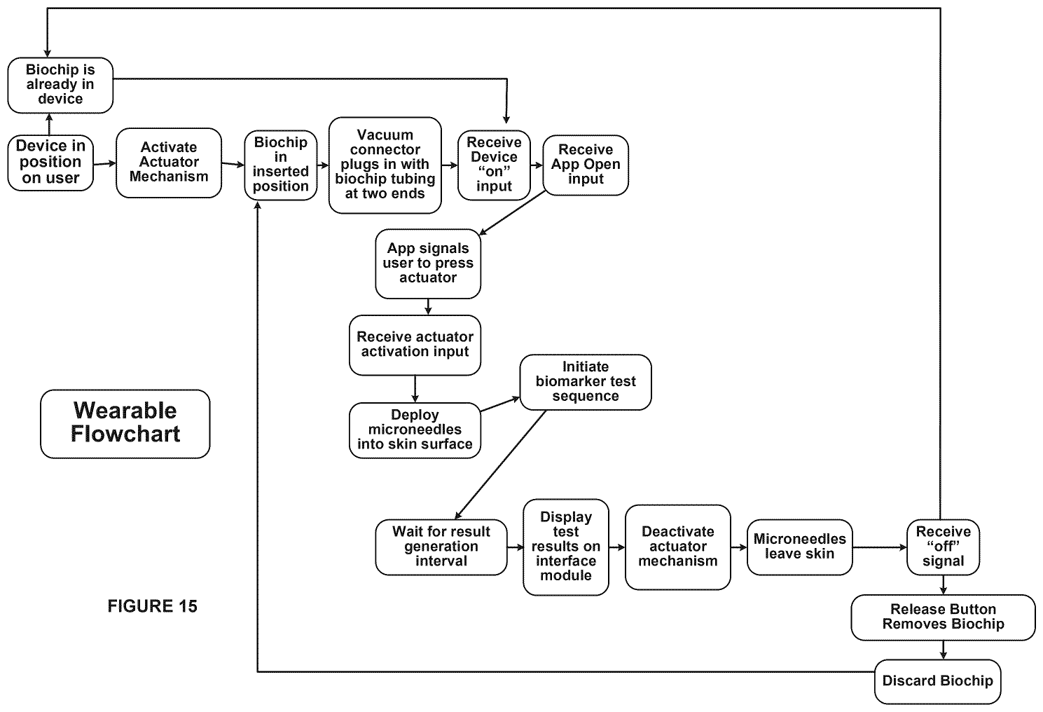

FIG. 15 illustrates a flowchart describing operational steps that may be employed when using the wearable diagnostic system incorporating the microfluidic assay (100) device (100), according to one embodiment of the present disclosure. The flowchart outlines a user workflow from device preparation to sample analysis and disposal.

Initially, the user may open the companion application and power on the wearable device (300). If a new biochip (100) is to be used, the user may retract the actuator (170), insert the disposable biochip (100) into the slot (240), and connect the vacuum tubing to both ends of the biochip (100). These steps may be skipped, for example, if a biochip (100) is already installed.

The user may then initiate the test via the app. When prompted by the app, the user may press the actuator (170), which deploys the microneedles (120) through the microneedle (120) interface hole (250) and into the skin. The microneedles collect a fluid sample, which may be analyzed by the biosensor (130). After approximately five minutes, test results appear in the app interface (310).

Following completion of the test, the user may turn off the device, retract the actuator (170), and press the release button (330) to remove the used biochip (100). The disposable biochip (100) may then be safely discarded. This stepwise process allows for easy, hygienic operation of the system and repeat testing with new biochips as needed.

FIG. 16A illustrates a top-down view of an exemplary microfluidic assay device (100), according to one embodiment of the present disclosure. The biochip (100) is shown without the topmost cover layer, exposing the internal sensor cavity (260) configured to house a biosensor platform. The cavity is centrally located within the chip body and may accommodate a detection substrate, such as a quartz crystal microbalance (QCM), impedance sensor, or other aptamer-functionalized surface for analyte detection. The cavity is fluidically connected to the microneedle array (120) and reagent reservoirs (110) via internal microchannels.

FIG. 16B illustrates a side view of the biochip (100) in one embodiment, showing the relative position of the microneedles (120) beneath the chip and a compression ridge configured to help secure and align the biochip within the wearable housing. The microneedles are shown extending downward for skin contact and fluid extraction, while the compression ridge facilitates stable mechanical coupling with the device's actuator interface or slot.

As shown across FIGS. 16A and 16B, the microneedles (120) are positioned to align with entry ports that lead directly into the sensor cavity (260), allowing extracted fluid to flow into the detection chamber. The cavity may include alignment features or conductive surfaces to interface with electrodes or data collection circuits.

This integrated layout supports modular biosensor replacement and provides a protected compartment for biomarker analysis, while preserving the biochip (100)'s disposable and compact form factor.

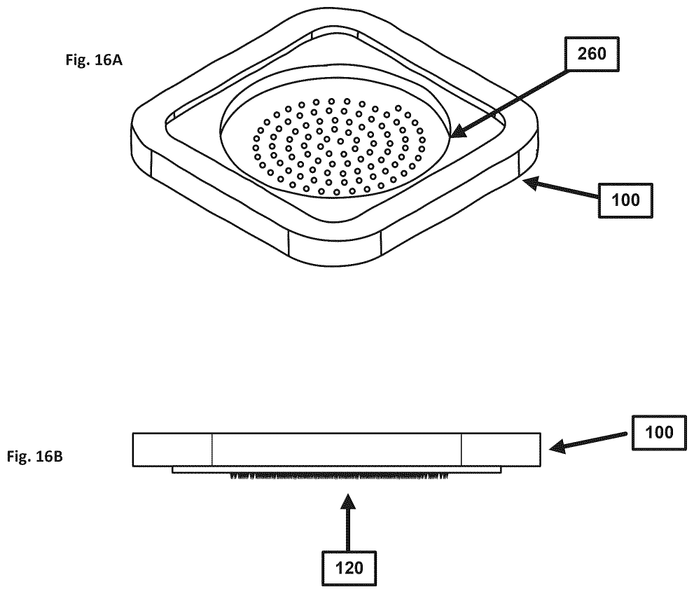

FIG. 17A provides a bottom isometric view of a biochip (100), according to one embodiment of the present disclosure. The view illustrates a full microneedle array (120) extending from the underside of the chip. The array may include multiple microneedles uniformly arranged to collect biological fluid such as interstitial fluid or capillary blood from the user in a minimally invasive manner.

FIG. 17B shows a magnified view of several the microneedle array.

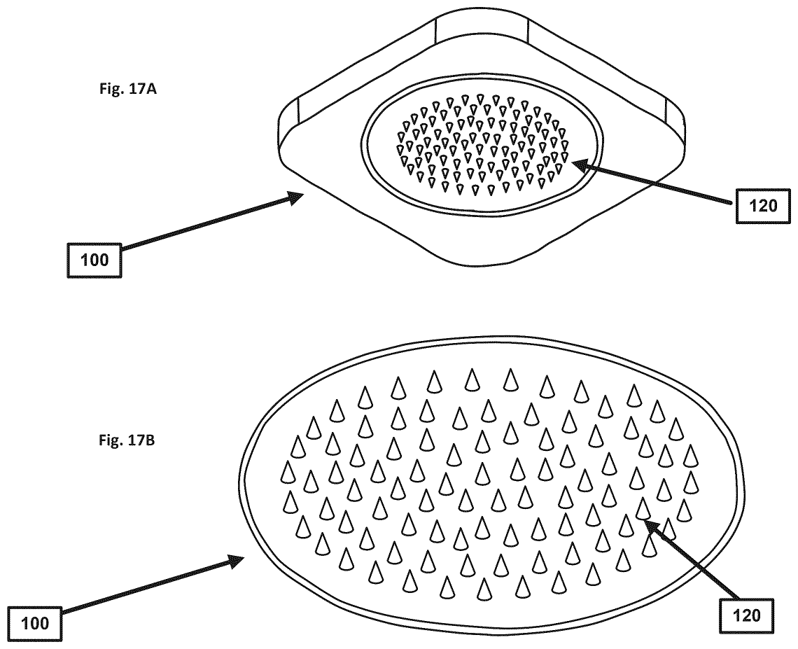

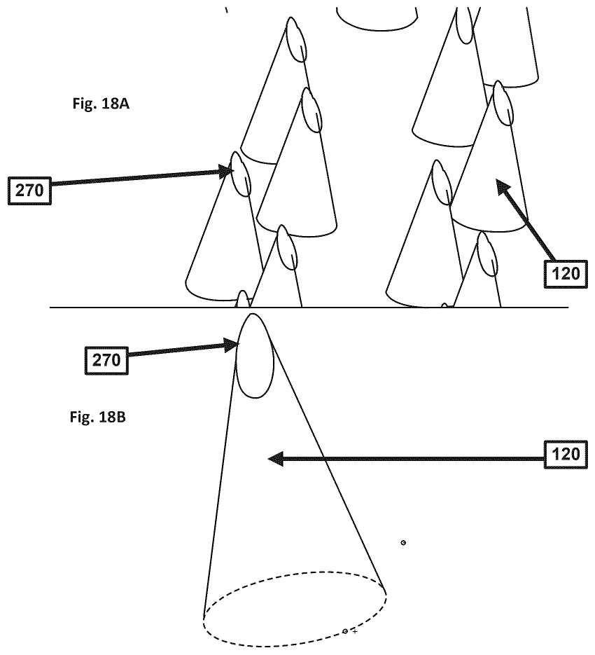

FIG. 18A provides a magnified perspective view of individual microneedles (120), highlighting their conical or tapered geometry, according to one embodiment.

FIG. 18B depicts an individual microneedles (120), highlighting its conical or tapered geometry, according to one embodiment. Each microneedle may have a base diameter of approximately 0.38 mm and a height of approximately 2.0 mm. These dimensions may be optimized for penetrating the stratum corneum without causing significant pain or bleeding.

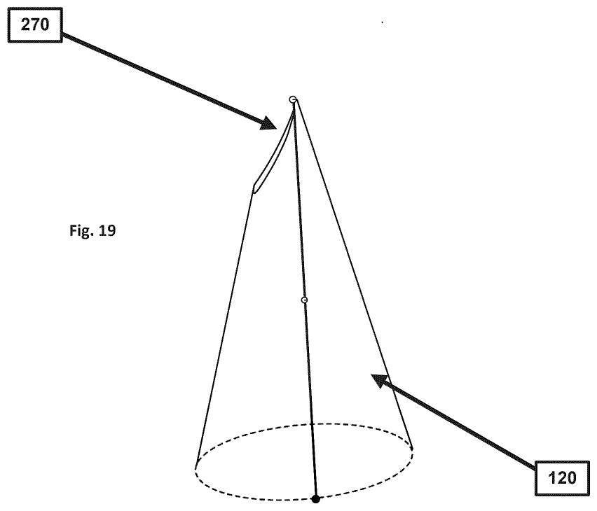

FIG. 19 shows a vertical dimensioned view of the micropore array and its alignment with the biosensor cavity (260). The diagram provides measured distances between micropores, cavity width, and alignment features that ensure uniform fluid delivery across the sensing surface.

FIG. 20 is a cross-sectional view of a micropore and its integration into the microchannel network (280). The section shows how biological fluid entering through the micropore (270) is directed into an underlying microchannel (280) for controlled routing to the biosensor cavity (260).

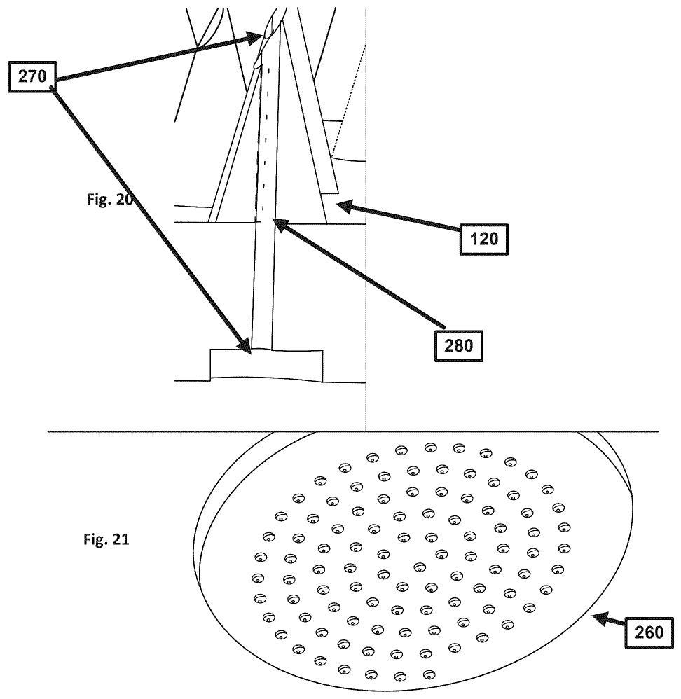

FIG. 21 provides a top view schematic of the micropore array as seen from above, showing each micropore (270) as an entry point into the fluidic network. This layout ensures that each microneedle (120) corresponds to a designated pathway, reducing cross-contamination and enabling analyte-specific detection.

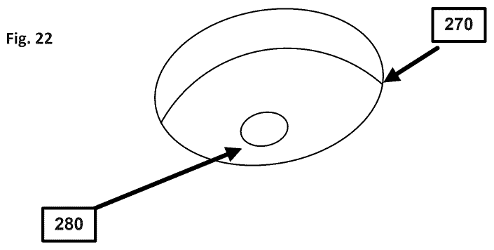

FIG. 22 shows a magnified top-down view of an individual micropore (270), highlighting its aperture and dimensions. The shape and size of the micropore are optimized to promote capillary action and minimize dead volume.

In one example, fluid movement within a microfluidic assay (100) can include the following steps. In one exemplary step, the microfluidic assay (100) is inserted, and the microfluidic assay (100) end tubes on both end sides of it align with the vacuum connectors located inside of the device. These tubes form a water tight seal when connected. In one exemplary step, microneedles strike the skin. In one exemplary step, the vacuum can be activated, pulling the blood from the skin into the microfluidic assay (100) and simultaneously pulling water from its reservoir, also located in the microfluidic assay (100), to where the blood is now being housed. In one example, the reservoir is located adjacent to the microfluidic assay (100). In one example, the microfluidic assay (100) is also referred to as the biosensor within the microfluidic assay (100).

In one exemplary embodiment, the vacuum can stop. In one exemplary embodiment, the blood can mix with the dilutant (water). In one exemplary step, the vacuum turns on. In one exemplary step, the blood can then be pulled onto the biosensor, which does the protein binding. In one example, the vacuum can stop, while the test is being done. In one exemplary step, after 5 minutes, the vacuum can turn on and pull the blood or water around the entire microfluidic assay (100). The blood or water can reenter the microfluidic assay (100) and can be stored in a waste reservoir. In one exemplary step, the vacuum can continue pulling fluid in intervals in order to reconfigure the biosensor for another test. This step is further explained under the cleaning step.

In an exemplary system, method, computer-accessible medium, and circuit for a microfluidic assay (100), pneumatic controlled microvalves can facilitate the flow of fluid. For example, air microfluidic channels that are controlled by the watch can control fluid flow within the microfluidic assay (100).

In one example, there can be two layers in the microfluidic assay (100). The top layer can be for air only, which is the pneumatic layer. At the bottom of this layer, there can be the top of the fluid layer. The bottom of this layer can have diaphragms that push downward into the fluid layer when pushed down by air pressure. This air pressure happens when the vacuum is activated. The diaphragm then can block certain fluid channels in the fluid layer. At the same time, the diaphragms can push fluids around in the microfluidic assay (100), while also restricting certain fluids via diaphragm blockages. In one exemplary embodiment, stationary microvalves can be located in the watch itself, instead of the biochip (100). The biochip (100) can then only have a fluid layer, and the top of it would have diaphragms that are pushed against it via mechanical force from the wearable. In one example, the rest of the process can work the same as far as fluid movement.

In one example, a PDMS diaphragm can be located on one of the lateral sides of the microfluidic assay (100). This PDMS diaphragm can interact with the wearables vacuum to allow the internal flow of fluids in the PDMS diaphragm.

In one example, the exemplary microfluidic assay (100) can include an opening that allows mechanical closing of the reagent valves. The physical barrier can originate from the wearable device (300) when the microneedle (120) actuator (170) located on the exterior of the wearable is pressed by the user. The physical barrier can press on a PDMS diaphragm that is acting as the reagent valve.

In one example, spring-loaded sections on the anterior end of the microfluidic assay (100) can allow for easy removal and insertion of microfluidic assay (100).

### Health Sensors

An exemplary system, method, computer-accessible medium, and circuit for a microfluidic assay (100) can be connected to a wearable device (300). The microfluidic assay (100) and/or the wearable device (300) can include one or more of the following:
Accelerometer, which can monitor movements and measure acceleration for tracking physical activity and workouts;
Optical heart rate sensor, which can measure heart rate and heart rate variability for tracking fitness and monitoring overall health;
Electrical heart sensor, which can use electrodes in the Digital Crown and back crystal to monitor heart rhythms and detect irregularities;
Gyroscope, which can measure rotation and orientation for accurate tracking of movements and activities;
Barometric altimeter, which can measure altitude and atmospheric pressure for tracking elevation changes during activities like hiking and climbing;
Ambient light sensor, which can automatically adjusts the screen brightness based on lighting conditions for better visibility;
GPS, which can use satellite signals to track location, distance, and route during outdoor workouts and activities;
Compass, which can help with navigation and tracking directional movements during activities;
Water resistance sensors, which can measure water pressure and detect when the watch is submerged in water for tracking swimming workouts;
Capacitive sensor, which can detect touch and pressure for various functions, such as taking ECG readings (on the Digital Crown and back crystal); and
Pulse oximeter, which can measure the saturation of oxygen in the blood for tracking overall health and fitness.

### Cleaning

An exemplary system, method, computer-accessible medium, and circuit for a microfluidic assay (100) can be reusable. The exemplary microfluidic assay (100) can extract (with microneedles) interstitial fluid or blood, to test the interstitial fluid or blood for the presence of biomarkers by selectively binding with aptamers that are affixed to a plate of conductive material such as gold, conductive carbon, or a conductive polymer; and to self-clean by removing bound biomarkers from previous tests from the aptamers, using either heat or reagents to remove the biomarkers, so that the biochip (100) can be reused. The valves where the reagents can leave their reservoir can be pressure activated by the vacuum to further control the timing of fluid release from the reservoirs. After the effectiveness of the aptamers has been exhausted, the aptamers can be removed from the gold plate on which they sit by circulating appropriate reagents, such as a piranha solution, over the gold plate. New aptamers can then be reattached through a sequential release of reagents that will link the aptamers to the gold plate, carbon plate or conductive polymer.

An exemplary system, method, computer-accessible medium, and circuit for a microfluidic assay (100) can be reusable. For example, the microfluidic assay (100) does not have to be removed to be reused. The microfluidic assay (100) can be cleaned inside of the microfluidic assay (100) through a series of vacuum on/off sequences. Subsequently, in one example, a software application connected to the microfluidic assay (100) can notify the user that the cleaning is complete and the microfluidic assay (100) is ready for reuse.

In one exemplary embodiment, the cleaning or self-cleaning step can include the following. In one exemplary step, blood can enter the biosensor and the target proteins are attached to the biosensors. In one exemplary step, after the mass is converted to a frequency and sent to the software application, the microfluidic assay (100) can be ready to be cleaned. In one exemplary step, EDTA or heat can dissociate the target protein from the aptamer. In one exemplary step, PBS can reconfigure the aptamers to their original structure. In one exemplary step, the microfluidic assay (100) can be ready to be used again.

An exemplary system, method, computer-accessible medium, and circuit for a microfluidic assay (100) can be reconfigured. In one example, new aptamers would be bound to the microfluidic assay (100) instead of disposing of it. The process can include the following steps. In one exemplary step, the microfluidic assay (100) was used to exhaustion. In one exemplary step, piranha solution diluted to 10% can be disposed from the reagent reservoir (110) to cover a biosensor. In one exemplary step, the aptamers can be destroyed on the sensor and the vacuum can move the fluid to a waste reservoir. The sequence of attaching new aptamers on the plate can include the following: avidin solution, phosphate buffered saline, dithiobis(succinimidyl propionate)) in Dimethylamine Solution, the aptamers, phosphate buffered saline, and Bovine serum albumin. The aptamers can now be usable and can be detectable for a different protein on the same electrode.

### Software Application

An exemplary system, method, computer-accessible medium, and circuit for a microfluidic assay (100) can collect frequency data from the microfluidic assay (100) and convert it into live data. An exemplary system, method, computer-accessible medium, and circuit for a microfluidic assay (100) can include a patient facing interface, which can include a dashboard. The dashboard can display the following exemplary information: health factors (which can be collected from health sensors) that result in a total score; health goals input and status; physician communication; and booking an appointment with a physician. In one example, the patient facing interface can display the following information: nutrition status, health sensor results (e.g., more specific information than what is presented in the dashboard), and lab tests results over time.

An exemplary system, method, computer-accessible medium, and circuit for a microfluidic assay (100) can include a physician facing interface, which can include a dashboard. The dashboard can include, e.g., patient Health alerts, patient profiles, and upcoming appointments. In one example the physician facing interface can include client communication; booking appointment for a patient; patient results; and lab tests results over time.

FIG. 14 shows an exemplary physician facing interface according to an exemplary embodiment of the present disclosure.

FIG. 15 is an exemplary flow chart for using the exemplary microfluidic assay (100) of the present disclosure.

The present disclosure is not to be limited in terms of the particular embodiments described in this application, which are intended as illustrations of various aspects. Many modifications and variations can be made without departing from its spirit and scope, as may be apparent. Functionally equivalent methods and apparatuses within the scope of the disclosure, in addition to those enumerated herein, may be apparent from the foregoing representative descriptions. Such modifications and variations are intended to fall within the scope of the appended representative claims. The present disclosure is to be limited only by the terms of the appended representative claims, along with the full scope of equivalents to which such representative claims are entitled. It is also to be understood that the terminology used herein is for the purpose of describing particular embodiments only, and is not intended to be limiting.

What is claimed is:

1. A microfluidic assay device comprising:
at least one microneedle configured to extract interstitial fluid, blood, or a combination thereof from a user;
a conductive region configured to receive fluid from the microneedle; and
at least one aptamer disposed on the conductive region, the aptamer being configured to selectively bind to one or more biomarkers present in the extracted fluid.

2. The device of claim 1, wherein the device is configured to remove a biomarker bound to the aptamer.

3. The device of claim 2, wherein the biomarker is removed from the aptamer by application of heat or one or more chemical reagents.

4. The device of claim 1, further comprising one or more valves configured to regulate the timing of fluid flow through the device.

5. The device of claim 4, wherein the one or more valves are actuated by a vacuum source.

6. The device of claim 4, further comprising a plurality of reservoirs configured to release reagents or air into the device in a time-controlled manner.

7. The device of claim 6, wherein the reservoirs are arranged in an alternating sequence of air-filled and reagent-filled reservoirs.

8. The device of claim 1, wherein the device is configured to refunctionalize the conductive region by applying one or more reagents that remove a used aptamer and sequentially attach a replacement aptamer.

9. The device of claim 8, wherein the conductive region comprises gold, carbon, or a conductive polymer.

10. The device of claim 1, further comprising a circuit configured to convert binding events between the aptamer and biomarker into digital data representing biomarker concentration.

11. The device of claim 1, further comprising an actuator configured to apply a mechanical force to the microfluidic assay device to drive the microneedle into the user's skin.

12. The device of claim 11, wherein the actuator is configured to engage with a recess or slot in the microfluidic assay device to align and position the actuator relative to the microneedle.

13. The device of claim 11, wherein the actuator is spring-loaded or vacuum-driven.

14. A system for biomarker detection comprising:
a wearable device;
a microfluidic assay device configured to be removably coupled to the wearable device, the microfluidic assay device comprising:
at least one microneedle configured to extract interstitial fluid, blood, or a combination thereof from a user;
a conductive region configured to receive the extracted fluid; and
at least one aptamer disposed on the conductive region and configured to selectively bind to one or more biomarkers present in the fluid; and
a processor within the wearable device configured to:
receive a signal corresponding to a binding event between the aptamer and the biomarker; and
generate data representing a concentration of the biomarker in the fluid.

15. A method for detecting one or more biomarkers in a fluid sample, the method comprising:
extracting interstitial fluid, blood, or a combination thereof from a user using at least one microneedle;
directing the extracted fluid to a conductive region of a microfluidic assay device; binding at least one biomarker in the fluid to at least one aptamer disposed on the conductive region;
generating a signal corresponding to the binding event; and processing the signal to determine a concentration of the biomarker.

16. The method of claim 15, wherein the microfluidic assay device is removably coupled to a wearable device.

17. The method of claim 15, further comprising transmitting the processed biomarker concentration data to a remote server, application, or physician interface.

18. The method of claim 17, further comprising generating an alert when the biomarker concentration exceeds a predetermined threshold and transmitting the alert to a physician.

19. The method of claim 15, further comprising establishing a baseline biomarker concentration for the user, and comparing subsequently measured concentrations to the baseline.

20. The method of claim 19, wherein the baseline biomarker concentration is established using a physician-defined reference value, an average of multiple prior uses by the user, or a population-based average adjusted for demographic or health factors.
